(19) United States
(12) Patent Application Publication
(10) Pub. No.: US 2017/0224797 A1
(43) Pub. Date: Aug. 10, 2017

(54) Cancer Therapy Using a Vaccine in Combination with a Cell-Based Immunotherapeutic Agent

(71) Applicant: Xeme Biopharma Inc., Lombard, IL (US)

(72) Inventors: Mircea C. Popescu, Plainsboro, NJ (US); Richard J. Robb, Gaithersburg, MD (US)

(21) Appl. No.: 15/428,871

(22) Filed: Feb. 9, 2017

Related U.S. Application Data
(60) Provisional application No. 62/293,506, filed on Feb. 10, 2016.

(51) Int. Cl. A61K 39/00; A61K 45/06; A61K 9/00

(52) U.S. Cl. CPC A61K 39/0011; A61K 9/0019; A61K 45/06; A61K 2039/54

(57) ABSTRACT

A cancer therapy composition and a method of cancer treatment by inducing humoral and cellular immune responses against cancer cells in a patient is provided. The method includes administering to the patient a therapeutically effective amount of a vaccine, wherein the vaccine includes at least one tumor-associated antigen, at least one immunostimulant, and at least one lipid capable of forming a multilamellar liposome, or non-lipid molecule capable of forming a vesicle or gel. A therapeutically effective amount of at least one cell-based immunotherapeutic agent is also administered to the patient before, after, or at the same time the vaccine is administered to the patient.

### RELATED APPLICATIONS

This application claims priority from U.S. Provisional Patent Application No. 62/293,506, filed Feb. 10, 2016, which is hereby incorporated herein by reference in its entirety for all purposes.

### FIELD

The technology described herein relates to methods for treating cancer and cancer vaccine compositions that include a cell-based therapeutic component. Specifically, this disclosure relates to methods of treating cancer using a cancer vaccine composition, that may be engineered from an individual patient's cancer, that includes natural or modified tumor-associated antigens combined with a cell-based therapeutic component that induces an active immune response to the patient's tumor.

### BACKGROUND

Efforts to treat patients with cancer utilizing the immune system dates back to the 1890 s. Cancer immunology and immunotherapy has advanced since then, and researchers have gained a better understanding of how the immune system identifies and attempts to destroy cancer cells. Researchers have also gained a better understanding of how cancers can undermine the immune system's ability to identify and destroy the cancer cells and significant progress has been made in the past decade in the treatment of cancer. Targeted forms of chemotherapy and various types of passive and active immunotherapy have improved clinical response rates, delayed disease progression, and prolonged survival.

Nevertheless, additional and less-toxic therapeutic modalities are needed to address the family of cancer diseases. Therapeutic vaccination, for example, aims to induce an active immune response to the patient's tumor and is an approach that holds particular promise for specificity and low toxicity, with the potential of long-term disease-free survival by activating the host's anti-tumor immune surveillance.

Several approaches have been used to induce active specific immunity including: 1) conventional vaccine formulations containing tumor antigens, see U.S. Pat. No. 6,312,718, Vaccine for B-Cell Malignancies incorporated herein by reference in its entirety for all purposes, 2) viral vectors which encode tumor antigens and/or immunostimulating agents, and 3) cell-based therapeutics consisting of activated antigen presenting cells, such as dendritic cells (“DC”) or tumor-targeted effector T lymphocytes. Approaches to cancer immunotherapy have been both patient specific, in which the therapy utilizes tumor biopsy material and immune cells derived from the individual patient being treated, and non-patient specific, which utilize tumor antigens common to a particular tumor type and cells derived from another individual or cell line. More recently, antibodies designed to interfere with tumor-induced immunosuppression, so-called checkpoint inhibitors, have shown success in the clinic. Despite this progress, more effective regimens for cancer immunotherapy are needed.

The present invention addresses the need for a more efficacious approach to cancer immunotherapy by optimally delivering three key elements for a tumor-specific immune response. Specifically, the invention combines a particulate formulation containing both tumor antigen and one or more immunostimulating agents together with a cell-based anti-tumor immunotherapeutic modality. Incorporation of tumor antigen into a liposomal vesicle or a gel is designed to enhance uptake by antigen presenting cells (“APC”). Inclusion of an immunostimulating agent, such as a cytokine or agonist for stimulatory receptors on APCs, amplifies the patient's endogenous immune response to the tumor antigen. Addition of an external cell-based tumor-specific modality serves to synergize with induction of the endogenous immune response. Thus, the combination approach described in the invention is designed to provide a more robust stimulation of active tumor-specific immunity and long-lived protection against cancer recurrence.

### SUMMARY

This Summary provides an introduction to some general concepts relating to this disclosure in a simplified form that are further described below in the Detailed Description. This Summary is not intended to identify key features or essential features of the disclosure.

Embodiments of the present disclosure are directed to a method of cancer treatment by inducing humoral and cellular immune responses against cancer cells in a patient that include the steps of 1) administering to the patient a therapeutically effective amount of a vaccine, wherein the vaccine includes at least one tumor-associated antigen, at least one immunostimulant, and at least one lipid capable of forming a multilamellar liposome, or non-lipid molecule capable of forming a vesicle or gel; and 2) administering to the patient a therapeutically effective amount of at least one cell-based immunotherapeutic agent.

Other embodiments of the present disclosure are directed to cancer therapy composition comprising 1) a cancer vaccine, wherein the cancer vaccine includes at least one tumor-associated antigen, at least one immunostimulant, and at least one type of lipid capable of forming a multilamellar liposome, or non-lipid molecule capable of forming a vesicle or gel; and 2) at least one cell-based immunotherapeutic agent. According to one aspect, the immunostimulant is selected from the group consisting of IFN-gamma, IL-2, IL-15, IL-23, M-CSF, GM-CSF, tumor necrosis factor, lipid A, CpG, CD80, CD86, and ICAM-1, or combinations thereof. According to other aspects, the cell-based immunotherapeutic agent is selected from the group consisting of dendritic cells, tumor-infiltrating T lymphocytes, chimeric antigen receptor-modified T effector cells directed to the patient's tumor type, B lymphocytes, natural killer cells, bone marrow cells, and any other cell of a patient's immune system, or combinations thereof.

Further features and advantages of certain embodiments of the present disclosure will become more fully apparent in the following description of embodiments and drawings thereof, and from the claims.

### DETAILED DESCRIPTION

In the following description of various examples of therapeutic cancer vaccines and related methods of treatment of the disclosure are described by example structures and environments in which aspects of the disclosure may be practiced. It is to be understood that other structures and environments may be utilized and that structural and functional modifications may be made from the specifically described structures and methods without departing from the scope of the present disclosure.

Aspects of the present disclosure are directed to a method of cancer treatment by inducing humoral and cellular immune responses against cancer cells in a patient comprising administering to the patient a therapeutically effective amount of 1) a vaccine, wherein the vaccine comprises at least one tumor-associated antigen, at least one immunostimulant, and at least one lipid capable of forming a multilamellar liposome, or non-lipid molecule capable of forming a vesicle or gel; and 2) administering to the patient a therapeutically effective amount of at least one cell-based immunotherapeutic agent. The liposome or non-lipid molecule can be substituted with any other delivery system known by those skilled in the art, such as systems made of cholesterol, cholesterol hemisuccinate or alpha-tochoferol (e.g., vitamin E), or other amphipathic molecules in which modified or synthesized cancer-associated antigens can attach or insert.

In another aspect, the vaccine and the immunotherapeutic agent are administered to the patient at a prescribed dose by intradermal, subcutaneous, intramuscular, intranodal, or intratumoral injection, or any combination thereof. In yet another aspect, the patient receives multiple vaccine and immunotherapeutic agent injections at prescribed time intervals. According to other aspects, the time intervals may include time intervals such as every 1, 2, 3, or 4 weeks or every 2 to 4 weeks. In still yet another aspect, the patient receives multiple vaccine and immunotherapeutic agent injections at different sites. In still yet another aspect, the patient receives multiple vaccine and immunotherapeutic agent injections at the same sites.

According to certain aspects, the immunotherapeutic agent is selected from the group consisting of dendritic cells, tumor-infiltrating T lymphocytes, chimeric antigen receptor-modified T effector cells, B lymphocytes, natural killer cells, bone marrow cells, and any other cell of an immune system, or combinations thereof. Immunotherapeutic agents or cell therapeutic components can include dendritic cells (DC) with or without prior in vitro maturation in the presence of the target tumor antigen, as well as in the presence of an appropriate cytokine(s). The cell therapeutic components may also include DCs with in vitro maturation and antigen loading using the cancer vaccine and appropriate factors. The addition of a single or multiple cell-based therapeutic components to the cancer vaccine helps overcome the factors produced my most cancers that act to directly suppress the patient's DCs and T cell functions, and other immunosuppressive effects related to the tumor. Such a method of cancer treatment with a cancer therapy composition that includes the cancer vaccine and at least one cell-based immunotherapeutic agent assists in the eliciting of an immune response that is not normally available to a patient with an immune system that is not intact. In other aspects, the immunotherapeutic agents include any antigen presenting cell.

According to another aspect, the immunotherapeutic agent is derived from an unrelated person. According to yet another aspect, the vaccine interacts with the immunotherapeutic agent in vitro before administering to the patient. According to another aspect, the vaccine and the immunotherapeutic agent are administered to the patient separately. According to yet another aspect, the vaccine and the immunotherapeutic agent are administered to the patient at the same time. According to another aspect, the vaccine and the immunotherapeutic agent are administered to the patient at different sites. According to other aspects, the vaccine and the immunotherapeutic agent are administered to the patient at the same sites.

The cell-based therapeutic component of the invention can take any of several forms as described earlier. Their preparation is familiar to those skilled in the art. Examples include dendritic cell DC therapies in which DC are removed from a patient, activated with a cytokine mixture and loaded with antigen in vitro and then returned to the patient to induce an immune response by the patient's T lymphocytes. Another form of cell therapy involves isolating T lymphocytes which have infiltrated the patient's tumor (TIL), expanding them in vitro and re-infusing them in the same patient, with or without added cytokines. Another example is Chimeric Antigen Receptor-modified T cell (CAR-T) therapies in which T cells are typically removed from a patient, modified with a vector that encodes a chimeric receptor targeting a cell surface molecule on the patient's tumor cells and then re-infused in the same patient. Other examples are isolation of B cells, NK cells or bone marrow cells from a patient, which may be followed by in vitro manipulation and/or expansion and re-infusion to the patient. There are also many variations of the above cell therapies, including forms that are not patient-specific.

According to certain aspects of the disclosure, the tumor-associated antigen is a synthetic tumor-associated protein or peptide. According to other aspects, the tumor-associated antigen is patient specific. In another aspect, the cancer vaccine includes a tumor-associated antigen that is identified by a genetic sequencing of the RNA (or DNA) contained in a hematologic tumor or a solid tumor-tissue sample obtained by needle biopsy, surgical excision, or other suitable method from one or more tumor sites of a patient. The genetic sequencing of a patient's tumor sample may be performed by techniques readily known to one skilled in the art or by using standard procedures, as described, for example, in U.S. Patent Publication No. 2011/0293637, Composition and Methods of Identifying Tumor Specific Neoantigens, incorporated herein by reference in its entirety for all purposes. According to another aspect, a subset of the tumor-associated antigens identified by genetic sequencing is then selected based upon their predicted affinity for binding to the individual patient's Major Histocompatibility Complex (MHC).

According to other aspects of the disclosure, the tumor antigenic component in the vaccine of the invention is any natural or synthetic tumor-associated protein or peptide or combination of tumor-associated proteins and/or peptides or glycoproteins or glycopeptides. In still yet other aspects, the antigenic component can be patient-specific or common to many or most patients with a particular type of cancer. According to one aspect, the antigenic component consists of a cell lysate derived from tumor tissue removed from the patient being treated. In another aspect, the lysate can be engineered or synthesized from exosomes derived from tumor tissue. In yet another aspect, the antigenic component consists of a cell lysate derived from tumor tissue extracted from one or more unrelated individuals or from tumor-cell lines.

The tumor-associated antigen component of the vaccine may be manufactured by any of a variety of well-known techniques. For individual protein components, the antigenic protein is isolated from tumor tissue or a tumor-cell line by standard chromatographic means such as high-pressure liquid chromatography or affinity chromatography or, alternatively, it is synthesized by standard recombinant DNA technology in a suitable expression system, such as E. coli, yeast or plants. The tumor-associated antigenic protein is then purified from the expression system by standard chromatographic means. In the case of peptide antigenic components, these are generally prepared by standard automated synthesis. Proteins and peptides can be modified by addition of amino acids, lipids and other agents to improve their incorporation into the delivery system of the vaccine (such as a multilamellar liposome). For a tumor-associated antigenic component derived from the patient's own tumor, or tumors from other individuals, or cell lines, the tumor tissue, or a single cell suspension derived from the tumor tissue, is typically homogenized in a suitable buffer. The homogenate can also be fractionated, such as by centrifugation, to isolate particular cellular components such as cell membranes or soluble material. The tumor material can be used directly or tumor-associated antigens can be extracted for incorporation in the vaccine using a buffer containing a low concentration of a suitable agent such as a detergent. An example of a suitable detergent for extracting antigenic proteins from tumor tissue, tumor cells, and tumor-cell membranes is diheptanoyl phosphatidylcholine. Exosomes derived from tumor tissue or tumor cells, whether autologous or heterologous to the patient, can be used for the antigenic component for incorporation in the vaccine or as a starting material for extraction of tumor-associated antigens.

Other embodiments of the present disclosure are directed to cancer therapy composition comprising 1) a cancer vaccine, wherein the cancer vaccine includes at least one tumor-associated antigen, at least one immunostimulant, and at least one type of lipid capable of forming a multilamellar liposome, or non-lipid molecule capable of forming a vesicle or gel; and 2) at least one cell-based immunotherapeutic agent. According to one aspect, the immunostimulant component in the cancer vaccine of the disclosure is any Biological Response Modifier (BRM) with the ability to enhance the therapeutic cancer vaccine's effectiveness to induce humoral and cellular immune responses against cancer cells in a patient. According to one aspect, the immunostimulant is a cytokine or combination of cytokines. Examples of such cytokines include the interferons, such as IFN-gamma, the interleukins, such as IL-2, IL-15 and IL-23, the colony stimulating factors, such as M-CSF and GM-CSF, and tumor necrosis factor. According to another aspect, the immunostimulant component of the disclosed cancer vaccine includes one or more adjuvant-type immunostimulatory agents such as APC Toll-like Receptor agonists or costimulatory/cell adhesion membrane proteins, with or without immunostimulatory cytokines. Examples of Toll-like Receptor agonists include lipid A and CpG, and costimulatory/adhesion proteins such as CD80, CD86, and ICAM-1.

According to one aspect, the immunostimulant is selected from the group consisting of IFN-gamma, IL-2, IL-15, IL-23, M-CSF, GM-CSF, tumor necrosis factor, lipid A, CpG, CD80, CD86, and ICAM-1, or combinations thereof. According to other aspects, the cell-based immunotherapeutic agent is selected from the group consisting of dendritic cells, tumor-infiltrating T lymphocytes, chimeric antigen receptor-modified T effector cells directed to the patient's tumor type, B lymphocytes, natural killer cells, bone marrow cells, and any other cell of a patient's immune system, or combinations thereof.

In one aspect, the cancer vaccine immunostimulant includes one or more cytokines, such as interleukin 2 (IL-2), GM-CSF, M-CSF, and interferon-gamma (IFN-γ), one or more Toll-like Receptor agonists and/or adjuvants, such as monophosphoryl lipid A, lipid A, muramyl dipeptide (MDP) lipid conjugate and double stranded RNA, or one or more costimulatory membrane proteins and/or cell adhesion proteins, such CD80, CD86 and ICAM-1, or any combination of the above. In one aspect, the cancer vaccine includes an immunostimulant that is a cytokine selected from the group consisting of interleukin 2 (IL-2), GM-CSF, M-CSF, and interferon-gamma (IFN-γ). In another aspect, the cancer vaccine includes an immunostimulant that is a Toll-like Receptor agonist and/or adjuvant selected from the group consisting of monophosphoryl lipid A, lipid A, and muramyl dipeptide (MDP) lipid conjugate and double stranded RNA. In yet another aspect, the cancer vaccine includes an immunostimulant that is a costimulatory membrane protein and/or cell adhesion protein selected from the group consisting of CD80, CD86, and ICAM-1.

The immunostimulant component of the vaccine is manufactured by standard means well-known to those skilled in the art. For cytokines and co-stimulatory or cell adhesion proteins, this typically involves recombinant DNA technology in a suitable host followed by standard chromatographic purification. For agonists of APC receptors, this typically involves synthetic chemistry or purification from a biological source.

According to other aspects of the disclosure, the disclosed cancer vaccine contains as a delivery system at least one lipid molecule capable of forming multilamellar liposomes or a non-lipid molecule capable of forming vesicles or a gel. In yet another aspect, the cancer vaccine includes a lipid molecule that is selected from the group consisting of phospholipids, glycolipids, cholesterol, and derivatives of the lipid molecules. In still yet other aspects, the lipid molecule is a saturated or an unsaturated phospholipid or glycolipid, or any combination of such molecules. In other aspects, the lipid molecule can include 1,2 dimyristoylphosphatidyl choline, 1,2 dipalmitoylphosphatidyl choline, 1,2 dimyristoylphosphatidyl glycerol, cholesterol, cholesterol hemisuccinate, alpha-tochoferol (e.g., vitamin E), and other derivatives of the above. According to another aspect, the non-lipid molecule or combination of molecules is any amphiphilic molecule capable of forming a vesicle or gel. According to still yet another aspect, the delivery system of the vaccine is composed of a combination of lipid and non-lipid molecules.

The liposome or particulate vesicle comprising the delivery system of the vaccine is formed by any of a number of standard methods, generally in the presence of the antigenic and immunostimulant components. In one example, hydrated DMPC phospholipid is combined with a sterile aqueous solution of tumor antigens and immunostimulatory agents, and subjected to repeated cycles of freezing, thawing and sonication. The resulting multilamellar liposomes incorporate the tumor antigens and the immunostimulatory agents within and between the lipid bilayers. The liposomes are typically in the range of 0.5 to 5 microns in diameter, a size suitable for uptake by APCs. Other methods for preparing liposomes, particulates or suitable gels are readily available to those skilled in the art. See e.g., U.S. Pat. No. 6,544,549, Multilamellar Coalescence Vesicles (MLCV) Containing Biologically Active Compounds, incorporated herein by reference in their entirety for all purposes).

According to other aspects, the composition further includes at least one checkpoint inhibitor. The inclusion of a checkpoint inhibitor prevents a patient's cancer tumor from using the associated checkpoints to protect themselves from immune system attacks. According to another aspect, treatment with the cancer therapy composition disclosed herein may be combined with co-administration of the checkpoint inhibitors. According to one aspect, the checkpoint inhibitors may include antibodies to the cell surface receptor PD-1, its ligand PD-L1, and antibodies to immune-inhibitory cell-surface proteins such as CTLA4. According to other aspects, the checkpoint inhibitors may be administered before the cancer therapy composition is administered. According to other aspects, the checkpoint inhibitors may be administered at the same time that the cancer therapy composition is administered. According to other aspects, the checkpoint inhibitors may be administered after the cancer therapy composition is administered.

Other embodiments of the disclosure relate to a cancer therapy composition that includes 1) a cancer vaccine, wherein the cancer vaccine includes at least one tumor-associated antigen, at least one immunostimulant, and at least one type of lipid capable of forming a multilamellar liposome, or non-lipid molecule capable of forming a vesicle or gel; and 2) at least one cell-based immunotherapeutic agent.

According to another aspect, the cancer therapy composition includes an immunostimulant selected from the group consisting of IFN-gamma, IL-2, IL-15, IL-23, M-CSF, GM-CSF, tumor necrosis factor, lipid A, CpG, CD80, CD86, and ICAM-1, or combinations thereof. According to yet another aspect, the lipid or lipid molecule is selected from the group consisting of saturated phospholipids, unsaturated phospholipids, glycolipids, cholesterol, alpha-tochoferol (e.g., vitamin E), and derivatives of thereof.

According to yet another aspect, the cancer therapy composition includes a cell-based immunotherapeutic agent is selected from the group consisting of dendritic cells, tumor-infiltrating T lymphocytes, chimeric antigen receptor-modified T effector cells directed to the patient's tumor type, B lymphocytes, natural killer cells, bone marrow cells, and any other cell of a patient's immune system, and combinations thereof. According to other aspects, the cell-based immunotherapeutic agent is derived from the patient. According to yet other aspects, the immunotherapeutic agent is derived from the patient. In still yet other aspects, the immunotherapeutic agent is derived from random individual. According to yet other aspects, the cancer vaccine interacts with the immunotherapeutic agent in vitro before administering to the patient. According to other aspects, the cancer therapy composition includes at least one checkpoint inhibitor.

According to another aspect, the cancer therapy composition is administered to the patient at a prescribed dose by intradermal, subcutaneous, intramuscular, intranodal, or intra-tumoral injection, or any combination thereof. According to another aspect, the patient receives multiple cancer therapy composition injections at separate sites or the patient may receive multiple cancer therapy composition injections at the same site. According to yet another aspect, the patient receives multiple cancer therapy composition injections at prescribed time intervals. According to other aspects, the time intervals may include time intervals such as every 1, 2, 3, or 4 weeks or every 2 to 4 weeks. According to other aspects, the immunotherapeutic agent is administered at different locations from the cancer vaccine injections. According to other aspects, the immunotherapeutic agent is administered at different times from the cancer vaccine injections.

Other embodiments of the disclosure relate to a cancer therapy composition that includes 1) a cancer vaccine, wherein the cancer vaccine comprises at least one tumor-associated antigen, at least one immunostimulant, and at least one type of lipid molecule capable of forming a multilamellar liposome, or non-lipid molecule capable of forming a vesicle or gel; 2) at least one cell-based immunotherapeutic agent selected from the group consisting of dendritic cells, tumor-infiltrating T lymphocytes, chimeric antigen receptor-modified T effector cells directed to the patient's tumor type, B lymphocytes, natural killer cells, bone marrow cells, and any other cell of a patient's immune system or combinations thereof; and 3) at least one checkpoint inhibitor.

An effective cancer therapy composition and related method of treatment by inducing humoral and cellular immune responses against malignant cells is described in this disclosure. The cancer therapy composition includes 1) a cancer vaccine, wherein the cancer vaccine includes at least one tumor-associated antigen, at least one immunostimulant, and at least one type of lipid capable of forming a multilamellar liposome, or non-lipid molecule capable of forming a vesicle or gel; and 2) at least one cell-based immunotherapeutic agent. Such a combination provides a novel and more potent vaccine formulation for treating cancer.

The cell-based immunotherapeutic agents described in this disclosure include any cell or combination of cells involved in the immune response. This combination provides a novel and more potent approach to cancer therapy by synergistically stimulating a response to the tumor. The combination therapy may be administered alone or together with a checkpoint inhibitor or similar agents that prevent tumor-induced immunosuppression.

As used herein, the terms “protein” and “polypeptide” and “peptide” are used interchangeably herein to designate a series of amino acid residues, connected to each other by peptide bonds between the alpha-amino and carboxy groups of adjacent residues. The terms “protein,” “peptide,” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “peptide” are used interchangeably herein when referring to a gene product and fragments thereof. Thus, exemplary polypeptides, peptides, or proteins include gene products, naturally occurring proteins, homologs, orthologs, paralogs, fragments and other equivalents, variants, fragments, and analogs of the foregoing.

An “antigen” is a substance that upon introduction into a vertebrate animal stimulates the production of antibodies or cell-mediated immune responses. A “tumor-associated antigen” is a molecule produced by or associated with malignant cells, but is not normally expressed, or expressed at very low levels, by a non-malignant cell. A “neoantigen” is class of tumor antigens that arises from tumor-specific mutations in an expressed protein.

Proteins or molecules of the “major histocompatibility complex (MHC)” are proteins capable of binding peptides that result from the proteolytic cleavage of protein antigens and representing potential T-cell epitopes, transporting them to the cell surface and presenting them there to specific cells, in particular cytotoxic T-lymphocytes or T-helper cells. The MHC of an individual's genome comprises the genetic region whose gene products expressed on the cell surface are important for binding and presenting endogenous and/or foreign antigens for regulating immune response. The major histocompatibility complex is classified into two gene groups coding for different proteins, namely molecules of MHC class I and molecules of MHC class II. The molecules of the two MHC classes are specialized for different antigen sources. The molecules of MHC class I present endogenously synthesized antigens, for example viral proteins and tumor antigens.

A “lipid” is any of a group of biochemicals which is variably soluble in organic solvents, such as alcohol. Examples of lipids include phospholipids, fats, waxes, and sterols, such as cholesterol. A “liposome” is a microscopic vesicle that consists of one or more lipid bilayers surrounding an aqueous compartment. See U.S. Pat. No. 6,544,549, Multilamellar Coalescence Vesicles (MLCV) Containing Biologically Active Compounds, incorporated herein by reference in its entirety for all purposes.

A “vaccine” is a material that is administered to a vertebrate host to immunize the host against the same material. Typically, a vaccine comprises material associated with a disease state, such as viral infection, bacterial infection, and various malignancies. A “therapeutic vaccine” is a vaccine administered to a vertebrate host which already has the disease being targeted and is designed to induce an immune response that causes disease regression, delayed disease progression, prolonged disease-free survival and/or overall survival.

An “immunostimulant” is any substance that stimulates the immune system by inducing activation or increasing activity of any of the immune system's components.

An “amino acid sequence” may be determined directly for a protein or peptide, or inferred from the corresponding nucleic acid sequence.

A “nucleic acid” or “nucleic acid sequence” may be any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

Definitions of common terms in cell biology and molecular biology can be found in The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present disclosure is performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); or Methods in Enzymology: Guide to Molecular Cloning Techniques Vol.152, S. L. Berger and A. R. Kimmel Eds., Academic Press Inc., San Diego, USA (1987); and Current Protocols in Protein Science (CPPS) (John E. Coligan, et. al., ed., John Wiley and Sons, Inc.), which are all incorporated by reference herein in their entireties.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The following examples are set forth as being representative of the present disclosure. These examples are not to be construed as limiting the scope of the present disclosure as these and other equivalent embodiments will be apparent in view of the present disclosure, figures and accompanying claims.

1. A method of cancer treatment by inducing humoral and cellular immune responses against cancer cells in a patient comprising
administering to the patient a therapeutically effective amount of a vaccine, wherein the vaccine comprises at least one tumor-associated antigen, at least one immunostimulant, and at least one lipid capable of forming a multilamellar liposome, or non-lipid molecule capable of forming a vesicle or gel; and
administering to the patient a therapeutically effective amount of at least one cell-based immunotherapeutic agent.

2. The method of claim 1, wherein the vaccine and the immunotherapeutic agent are administered to the patient at a prescribed dose by intradermal, subcutaneous, intramuscular, intranodal, or intratumoral injection, or any combination thereof.

3. The method of claim 1, wherein the patient receives multiple vaccine and immunotherapeutic agent injections at different sites.

4. The method of claim 1, wherein the patient receives multiple vaccine and immunotherapeutic agent injections at prescribed time intervals.

5. The method of claim 1, wherein the immunotherapeutic agent is selected from the group consisting of dendritic cells, tumor-infiltrating T lymphocytes, chimeric antigen receptor-modified T effector cells, B lymphocytes, natural killer cells, bone marrow cells, and any other cell of an immune system.

6. The method of claim 1, wherein the immunotherapeutic agent is derived from the patient.

7. The method of claim 1, wherein the immunotherapeutic agent is derived from an unrelated person.

8. The method of claim 1, wherein the vaccine interacts with the immunotherapeutic agent in vitro before administering to the patient.

9. The method of claim 1, wherein the vaccine and the immunotherapeutic agent are administered to the patient separately.

10. The method of claim 9, wherein the vaccine and the immunotherapeutic agent are administered to the patient at different sites.

11. The method of claim 1, wherein the tumor-associated antigen is a synthetic tumor-associated protein or peptide.

12. The method of claim 1, wherein the tumor-associated antigen is patient specific.

13. The method of claim 1, wherein the vaccine is administered with a checkpoint inhibitor.

14. A cancer therapy composition comprising
a cancer vaccine, wherein the cancer vaccine comprises at least one tumor-associated antigen, at least one immunostimulant, and at least one type of lipid capable of forming a multilamellar liposome, or non-lipid molecule capable of forming a vesicle or gel; and
at least one cell-based immunotherapeutic agent.

15. The composition of claim 14, wherein the immunostimulant is selected from the group consisting of IFN-gamma, IL-2, IL-15, IL-23, M-CSF, GM-CSF, tumor necrosis factor, lipid A, CpG, CD80, CD86, and ICAM-1.

16. The composition according to claim 14, wherein the lipid molecule is selected from the group consisting of saturated phospholipids, unsaturated phospholipids, glycolipids, cholesterol, alpha-tochoferol, and derivatives of thereof.

17. The composition according to claim 14, wherein the cell-based immunotherapeutic agent is selected from the group consisting of dendritic cells, tumor-infiltrating T lymphocytes, chimeric antigen receptor-modified T effector cells directed to the patient's tumor type, B lymphocytes, natural killer cells, bone marrow cells, and any other cell of a patient's immune system.

18. The composition according to claim 14, wherein the cell-based immunotherapeutic agent is derived from the patient.

19. The composition according to claim 14, wherein the vaccine interacts with the immunotherapeutic agent in vitro before administering to the patient.

20. The composition according to claim 14, wherein the composition further includes a checkpoint inhibitor.

21. A cancer therapy composition comprising
a cancer vaccine, wherein the cancer vaccine comprises at least one tumor-associated antigen, at least one immunostimulant, and at least one type of lipid molecule capable of forming a multilamellar liposome, or non-lipid molecule capable of forming a vesicle or gel;
at least one cell-based immunotherapeutic agent selected from the group consisting of dendritic cells, tumor-infiltrating T lymphocytes, chimeric antigen receptor-modified T effector cells directed to the patient's tumor type, B lymphocytes, natural killer cells, bone marrow cells, and any other cell of a patient's immune system; and
at least one checkpoint inhibitor.
